(19) United States
(12) Patent Application Publication
(10) Pub. No.: US 2004/0167429 A1
(43) Pub. Date: Aug. 26, 2004

(54) Cutaneous biopsy device with handle and disposable tips

(76) Inventors: Babak B. Roshdieh, Fresno, CA (US); Martin Edward Smith, Camarillo, CA (US)

Correspondence Address: KENNETH J. HOVET, NORDMAN, CORMANY, HAIR & COMPTON, P.O. BOX 9100, 1000 TOWN CENTER DRIVE, OXNARD, CA, 93031-9100, US

(21) Appl. No.: 10/371,277

(22) Filed: Feb. 20, 2003

(51) Int. Cl. A61B 10/00

(52) U.S. Cl. 600/567

(57) ABSTRACT

A skin punch device comprises a gripping handle with a center passage, through which a reciprocating plunger extends. A disposable cutting tip is releasably connected to the handle. The cutting tip has a tip cavity with a base from which extends a biopsy chamber that terminates at cutting edges. The base has an aperture through which a slidable push-rod extends. The push-rod has a disc end located in the biopsy chamber. The plunger and push-rod are in axial alignment, so that when the plunger is moved, the push-rod will also move in tandem. Movement of the push-rod will simultaneously cause the disc end to pass through the biopsy chamber and eject a cut tissue sample lodged in the chamber. Further movement of the plunger will result in engagement with the tip cavity and dislodgement of the entire disposable cutting tip from the end of the gripping handle.

### BACKGROUND OF THE INVENTION

1. Field of the Invention

The present invention relates to the field of biopsy tools. More specifically, the invention relates to a biopsy cutting tool comprising a re-usable handle and a disposable tip for obtaining cutaneous tissue samples.

2. Description of Related Art

Advances in modern medicine rely increasingly on advanced diagnostic procedures to identify pathogens and diseases at an early stage. One of the more important diagnostic techniques is the securing of a tissue sample for biopsy. A cutaneous biopsy is a skin sample taken for a variety of purposes, including diagnosing the presence of melanoma, carcinoma, and other forms of skin cancer.

A tool for taking a cutaneous tissue sample comprises a cutting edge formed along an outer edge of a cylindrical barrel. The cutting edge is pressed against tissue and the barrel is rotated, cutting into the tissue and obtaining a cylindrically shaped tissue sample.

In order to retrieve the tissue sample from the cylindrical barrel, in the past, physicians or lab technicians have been known to skewer the tissue sample with a needle and pry it out of the barrel shaped blade. In light of the possibility of cutting oneself on the contaminated cutting blade while trying to retrieve the tissue sample, it can be readily appreciated that such a procedure has potentially grave health risks to the lab technician or physician.

U.S. Pat. No. 3,692,020 to Schied teaches a gun-shaped housing within which is positioned a hollow spindle to which is attached a ratchet mechanism actuated by a trigger. An ejector rod is biased outwardly by a spring which extends through the spindle. At the rod's inner end is located a disk, which is positioned within the upper end of the cutting die. In operation, the spindle is wound-up by the knob against the bias torque spring. Upon actuation of the trigger, the spring will cause a reverse unwinding which will rotate the cutter to induce a cutting action against the tissue. Upon completion of the cutting action, the die is lifted form the skin and the ejector rod is depressed. The depression of the ejector rod forces the disk against tissue sample, thereby ejecting the specimen from the die interior. Schied, however, does not teach a disposable blade and tip, and re-sterilization must be performed after each use.

U.S. Pat. Nos. 5,183,053 and 5,186,178 to Yeh et al. discloses a disposable handle in which a disposable blade is pressed fitted. The entire unit is then discarded after use. Yea et al. do not teach a reusable handle. Additionally, Yeh et al. do not teach a means for ejecting the tissue safely from the elliptically shaped blade after securing the biopsy sample, and the lab technician is forced to remove the tissue sample by some makeshift means.

U.S. Pat. No. 5,833,628 to Yuan et al. teaches a disposable bone graft harvester having a clear, graduated plastic tube tipped with a bone cutting head at one end and a handle or other torque supplying connection at the other end. An ejector rod having an enlarged disc-like end is fitted within a tissue-receiving sleeve. At the end of the sleeve is a cutting head comprising rotating blades which remove bone samples which are then accumulated in the sleeve. The cutting head is subsequently removed, and the ejector rod is depressed to deposit the cut tissue into a selected receptacle.

Yuan et al. is not directed to a cutaneous biopsy device, but rather, to a bone graft harvester. Instead of securing a single contiguous tissue sample, Yuan et al has rotating blades that scrape away small flecks of bone with each rotation. Moreover, Yuan et al. does not teach a re-usable handle which does not need to be subjected to re-sterilization after each use.

U.S. Pat. No. 6,155,989 to Collins discloses a syringe-like disposable cutaneous biopsy instrument equipped with a tubular blade at its lower end, and designed so that a vacuum is created during use. The vacuum serves to retain undeformed plug of tissue cut from a patient's skin. Collins does not teach a reusable member. He also does not teach a second plunger that is separated from contaminated fluids and tissue.

U.S. Pat. No. 6,440,086 to Hohenberg describes an internal biopsy device comprising a needle for piercing tissue and attaining a desired depth for an internal biopsy. Hohenberg is not directed to a cutaneous biopsy device, and he does not teach a cutting head which is removable by actuating a plunger.

### SUMMARY OF THE INVENTION

The biopsy device of the present invention overcomes the aforementioned prior art disadvantages by providing a cutting means that is readily removed from a handle means and is disposable. The cutting means further includes a discharging means for ejecting tissue samples into a storage receptacle. A coupling means is also provided to releasably attach the cutting means to the handle means. Additionally, the handle means includes a release means that advantageously allows rapid release of the cutting means from the handling means.

More particularly, the device comprises a handle to which is attached a plunger and a disposable cutting tip member. The handle comprises an elongated structure having a longitudinal center passage that extends from an open upper end to a lower discharge end. The plunger is in sliding engagement with inner surface portions of the center passage and includes an inner plunger head that is spaced above the free end of a push rod that moves axially within an aperture in the tip member. The tip member includes a base from which extend tip sidewalls that define an open tip cavity. Extending from the base in a direction opposite from the tip sidewalls, are cutter walls. The cutter walls terminate at sharp cutting edges which are used to cut into a patient's skin. The cutter walls define a biopsy chamber for temporarily housing a skin biopsy sample prior to being ejected into a storage receptacle.

The base is provided with a center aperture through which extends the push rod. The push rod comprises a shaft with a free end which extends into the tip cavity. At the shaft's opposing end is an enlarged disc having a periphery that corresponds to the cross-sectional shape of the biopsy chamber.

The tip cavity sidewalls releasably engage the handle discharge end, and includes an abutment means for engaging the plunger contact end (head) to cause dislodgement of the tip member when the plunger is moved to a fully depressed position. Prior to engagement with the abutment means, the plunger will be in an intermediate position whereby the plunger contact end will engage the push rod free end and cause the opposing disc end to move axially into the biopsy chamber. The opposing disc end functions to displace any biopsy contents that may be in the chamber as a result of tissue cutting during operation of the device.

The plunger has an actuating end that projects outwardly beyond the handle's open upper end to permit manual depression of the plunger. Axial alignment of the plunger is provided by its sliding engagement with an interior reduced diameter section of the center passage. The center passage includes a compression spring to bias the plunger outwardly in a non-operative stowable position.

The handle discharge end is provided with tip attachment means for releasably engaging the tip member. It comprises an outwardly extending annular connecting structure with connector walls having an outer engagement surface that may be tapered. The cross-sectional shape of the connecting structure corresponds to the cross-sectional shape of the tip cavity sidewalls. This arrangement permits the formation of a friction engagement joint between the tip cavity inner surfaces and the connecting structure outer engagement surfaces. Alternative tip engagement means are offsetting annular beads, or an annular bead on one surface with a corresponding annular groove on the opposing surface, or axially aligned ribs, serrations and dimples on one or both of the joint surfaces. An elastic O-ring or other resilient friction engagement structure could also be utilized between the joint surfaces to effect a releasable connection.

The present invention further comprehends the use of a tray means to display an array of cutting tip members having a variety of cutting edge shapes and sizes. The tip members are oriented in the tray means with respective tip cavities uppermost. In this orientation, a user can dislodge and discard a used tip member and subsequently engage a selected fresh tip member. This is conveniently accomplished by positioning the handle tip attachment means over the tip cavity of the selected tip member and pushing the handle down until the joint surfaces are effectively engaged. The tray means may include compartments for holding different sizes and types of tip members. The entire tray means will be sterilized and overlaid with a sealed covering. Alternatively, each compartment can be overlaid with an individual cover to avoid contamination of adjacent compartments.

The invention also provides a method of operating a cutaneous tissue-sampling device having a cutting edge formed on the edge of a substantially cylindrical member. The method comprises the steps of pressing a cutting edge of the cutaneous tissue-sampling device against a tissue, wherein a handle of the cutaneous tissue-sampling device is oriented on a conceptual plane at a reference angle of zero degrees, rotating the cutaneous tissue-sampling device around an axis of the reference angle while maintaining pressure against the tissue; depressing the cutting edge into the tissue, thereby cutting a tissue sample within the substantially cylindrical member; angling the handle of the cutaneous tissue-sampling device to a positive angle of orientation on the conceptual plane; rotating the cutaneous tissue-sampling device around an axis of the positive angle of orientation, thereby severing the tissue sample from an underlying connective tissue; and withdrawing the cutaneous tissue-sampling device. The above actions will permit the tissue to remain lodged within the substantially cylindrical member of the tissue sampling device. As such, a significant problem in the prior art has been overcome wherein prior attempts to lift the sample from surrounding tissue by forceps, toothpicks and the like, caused damage to the tissue.

At least the biopsy chamber of the tip member may be transparent. This feature helps a user in making an accurate biopsy cut. Also, it allows the use of gradations which indicate a depth of tissue cut by the blade.

The cutting edges may define a curved or polygonal opening. The cutting edge may be constructed of a moldable plastic or resin material to produce a cutting edge of surgical quality without the use of machine tooling or abrasion.

### BRIEF DESCRIPTION OF THE DRAWINGS

FIG. 1 is an isometric view of a cutaneous biopsy device comprising a reusable handle and a disposable tip.

FIG. 2 is a cross-sectional view taken along lines 2-2 of FIG. 1.

FIG. 3 is a fragmentary cross-sectional view of the front portion of the FIG. 1 device depicting dislodgement of a biopsy specimen.

FIG. 4 is a view similar to FIG. 3 depicting the beginning of disengagement of the disposable tip.

FIG. 5 is a view similar to FIG. 3 illustrating complete disengagement of the disposable tip for the handle.

FIG. 6 is a top plan view of a package containing disposable tips.

FIG. 7 is an enlarged cross-sectional view taken along line 7-7 of FIG. 6.

FIG. 8 is an enlarged cross-sectional view of a transparent disposable tip.

FIG. 9 is an enlarged fragmentary cross-sectional view of the tip separated from the handle wherein frictional engagement occurs with a bead and groove joint.

### DESCRIPTION OF THE PREFERRED EMBODIMENTS

The present invention is directed to a cutaneous biopsy device comprising a disposable tip 30 coupled to a re-usable handle 10. The handle includes a plunger 27 and the tip includes a push rod 33 that interacts with the plunger during operation of the device.

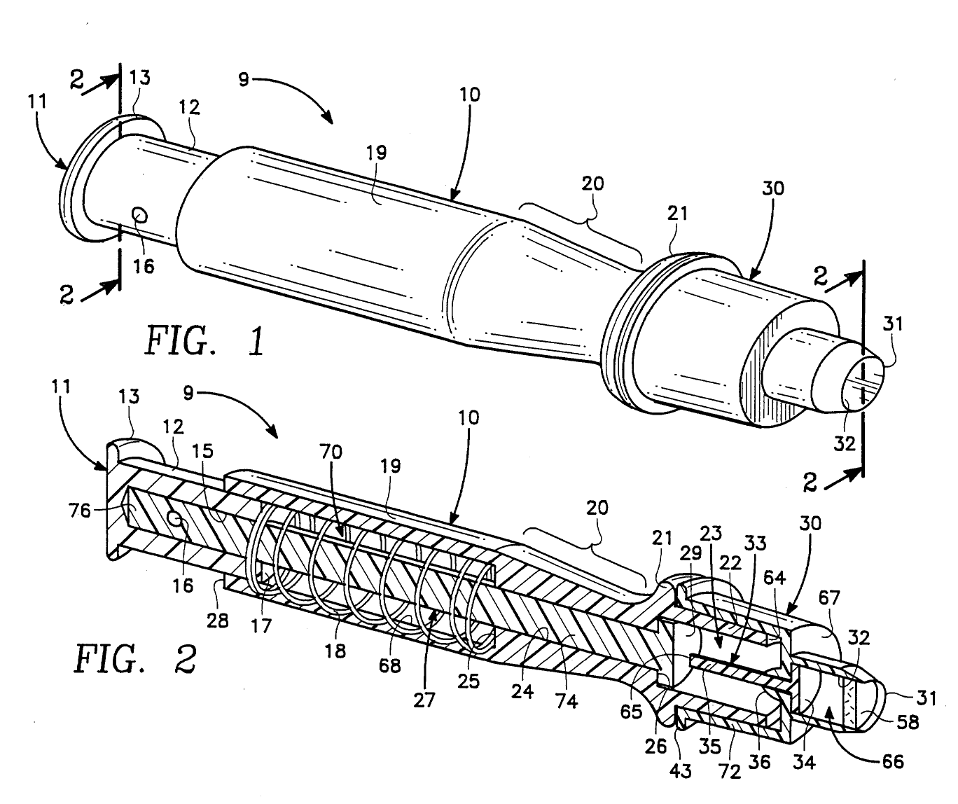

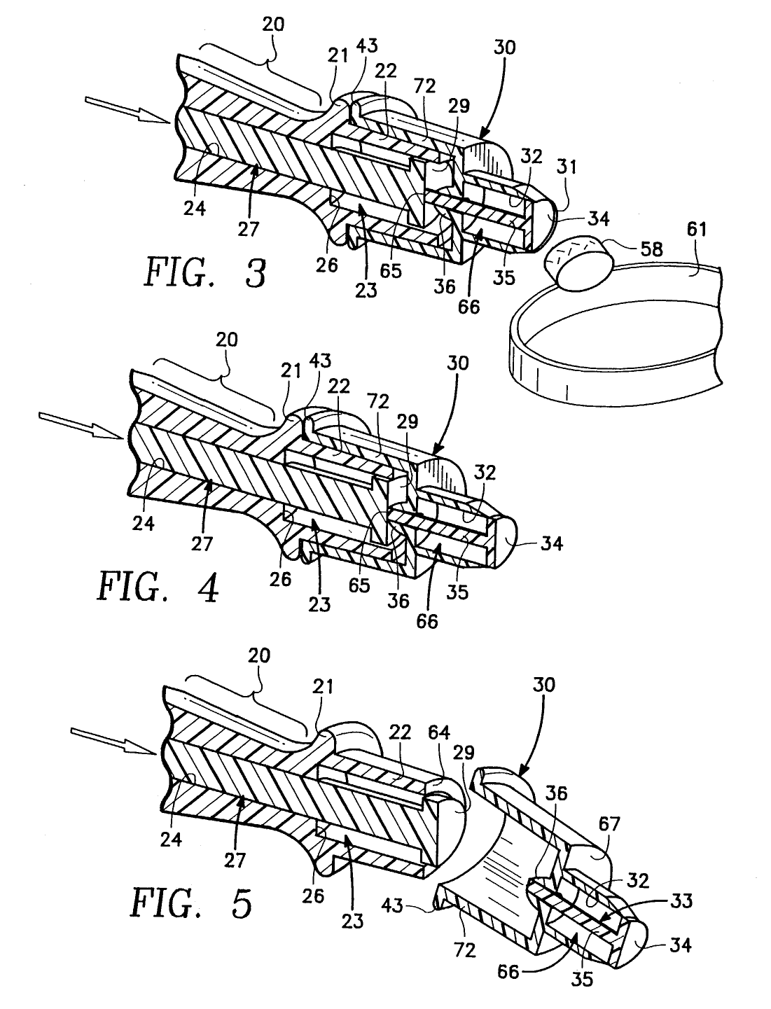

FIG. 1 illustrates a fully assembled biopsy device. FIG. 2 is a cross-sectional view of the biopsy device of FIG. 1 illustrating the component parts fitted together and interacting as a unit. FIGS. 3-5 illustrate operation of the device.

With reference now to FIGS. 1 and 2, handle 10 includes a grip region 19. Formed within the grip region is a longitudinal center passage 24. The passage extends from an open upper end 28 to a lower discharge end 64. The plunger 27 is in sliding engagement with inner surface portions of the center passage 70, and includes an inner contact head 29 that is spaced above free end 65 of push-rod 33 that moves axially within the cutting tip member 30. The tip member comprises a base 67 from which extend tip sidewalls 72 that define an open tip cavity 23.

Extending from base 67 in a direction opposite from the tip sidewalls, are cutter walls 32. The cutter walls terminate at sharp edges 31, which are used to cut into a patient's skin. The cutter walls define a biopsy chamber 66 for temporarily housing a skin biopsy specimen 58 upon completion of the cutting process.

The center passage includes an enlarged portion 68 extending from handle open end 28 to inner ledge 25. The longitudinal extent of the large portion corresponds generally to the length of grip region 19. The upper channel ledge 25 creates the beginning of a reduced diameter section 24 of the central passage. This section further extends toward the lower handle discharge end 64, until it merges into a radially outwardly extending shoulder 26. The connecting wall outer surfaces may optionally be tapered to provide a frictional engagement with the inner surfaces of the tip cavity sidewalls 72.

The reusable plunger 27 slidably fits within the center passage 70. It comprises a plunger shaft 74 having a lower end that merges into plunger head 29. The plunger upper end is fastened to a button 11. The plunger shaft has a diameter that is slightly less than the reduced diameter section 24 of the center passage. The plunger head 29 has a disc-like shape with a diameter less than the interior diameter of handle connector walls 22. The plunger has sufficient length to position the plunger head 29 within tip cavity 23 while also extending to an attachment end 76 beyond handle open end 28.

Button 11 is a structure having an actuating head 13 from which extends a body portion 12. The body portion terminates at inner edge 17. Extending axially into the inner edge is a button bore 15. Attachment end 76 of the plunger shaft extends into the button bore and is fastened to body portion 12 with securing pin 16 in a manner known in the art.

A compression spring 18 is disposed within the center passage enlarged portion 68. The spring has a lower end that rests against enlarged passage ledge 25, and an upper end that abuts against button inner edge 17. When in a first stowable position as shown in FIG. 2, the spring will be uncompressed and the button actuating head 13 will be spaced above handle open end 28. As the actuating head is depressed, the spring 18 will exert a counter force against button inner edge 17 and shoulder 25, thereby resisting depression of the button. The button 11 and plunger 27 thereby advance and retract as a single unit, as described below.

As the spring 18 forces button 11 upward to the first position, the reusable plunger also moves upward until plunger head 29 engages shoulder 26 formed at the junction of connector wall 22 and the reduced diameter section 24. This engagement prevents further retraction of the plunger. It also prevents the button from moving beyond handle open end 28. This arrangement further ensures that, when pressure is not being exerted against the button, the plunger head 29 will not inadvertently advance to exert pressure on push rod 33, nor on boss 36 of the disposable tip 30. As discussed below, this is important in that it ensures that a tissue sample will not be prematurely ejected from the biopsy chamber, and that the tip member 30 will not be prematurely separated from the handle.

The handle 10 is ergonomically designed to fit the human hand, and includes a grip region 19 that merges into a tapered section 20. The tapered section terminates at finger interface flange 21. The tapered section 20 and the finger interface flange 21 afford a user various ways to grip the cutaneous biopsy device during use, including placing the index finger and middle finger below the interface flange 21 like a syringe and activating the button by the force of one's thumb, or by wrapping one's fingers around the grip region, little finger adjacent to the interface flange 21, and thumb pointing up and positioned to depress button 11.

Preferably, the tip member 30 comprises a tip cavity 23 and biopsy chamber 66 that are cylindrical. The tip cavity is defined by a base 67 from which extend tip sidewalls 72. The tip sidewalls extend upwardly to an outwardly flared lip 43. As best shown in FIGS. 1 and 2, when the tip member is fully engaged with handle connector walls 22, the lip 43 will abut against the underside of interface flange 21.

With reference to FIG. 8, extending through the base 67, coextensive with the cavity center axis, is a rod aperture 37. Preferably, the base includes an inwardly extending boss 36 through which the rod aperture also extends. The boss adds length to the rod aperture for greater lateral strength. It also provides an abutment means for engagement with plunger head 29 during operation of the device.

Extending outwardly from below base 67 is a cutter wall 32. The cutter wall is coaxial with the device longitudinal center axis and terminates at cutting edge 31.

A disposable push rod 33 comprising an enlarged disc part 34 and a rod shaft 35, is oriented within the tip member such that the disc part is in biopsy chamber 66, and the rod shaft passes through rod aperture 37 and into tip cavity 23.

The diameter of the rod aperture is preferably almost the same diameter as rod shaft 35. The relative diameters permit the rod shaft to move through the rod aperture in a smooth snug alignment with the device center axis. The snug fit also prevents bodily fluids from leaking through the rod aperture and contaminating reusable parts of the device. Additionally, having a snug fit will inhibit the push rod from sliding entirely out of the rod aperture during disengagement of the biopsy specimen 58.

Under magnification, virtually all cutting edges are seen to be less than perfectly sharp. At a certain point of magnification, cutting edges typically comprise jagged and rough edges. The level of magnification necessary to reveal these imperfections is a measure of the quality of a cutting edge. A cutaneous biopsy device, such as the present invention, utilizes a round cutting edge that is pressed into skin, rotated and swivelled to sever the skin tissue at a selected depth. To detach the specimen from underlying tissue, the blade must also be tilted. Those skilled in the art will understand that, much like a paper cut, any cutting edge designed to be moved across tissue, need not be as sharp as a cutting edge that must cut tissue by pressure only. Thus, use of a moldable plastic or resin material satisfies industry requirements for biopsy cutting blades.

Moreover, providing a tip member made of plastic allows for the use of a clear plastic material such as polycarbonate and acrylic polymers. This feature of the invention allows a physician or medical practitioner to more easily see the progress of cutting a tissue sample. In particular, it permits a user to better undercut a tissue specimen at a selected depth. FIG. 8 illustrates transparent tip member 38 with gradation lines 59 across the outer surface of cutter wall 32. As a user penetrates a patient's skin with cutting edge 31, the depth of penetration can be determined by observing the upward movement of tissue into the biopsy chamber in relation to the predetermined gradation lines.

FIG. 3 illustrates the discharge of a tissue sample 58 from biopsy chamber 66. Following completion of the tissue cutting step, the plunger 27 and push rod 33 will be in a first position as shown in FIG. 2. Thereafter, the biopsy chamber containing the cut tissue sample 58, will be positioned over tissue receptacle 61 for receiving and storing tissue samples. Subsequently, button 11 is pressed a first distance to a second intermediate position as shown in FIG. 3. The first distance is no less than the axial length of the biopsy chamber. This movement will engage plunger head 29 with push rod free end 65. Continued movement will advance disc part 34 to the outermost open end of the biopsy chamber, defined by cutting edge 31. During the above movements, the disc part will engage the cut tissue sample and push it out of the biopsy chamber, thereby allowing the sample to drop into the aforementioned receptacle 61.

Initially, it will be appreciated that the overall length of the push rod 33 will be no less than the axial length of the biopsy chamber. This is necessary to completely expel the tissue sample from the biopsy chamber during movement through the aforementioned first distance to the second intermediate position as shown in FIG. 4.

Further movement of button 11 from the second position will cause plunger head 29 to engage base 67 of the tip cavity 23. Optionally, boss 36 will be contacted as shown in FIG. 4. Continued axial movement by button 11 will release the friction joint between the outer surface of handle connector wall 22 and the inner surfaces of tip cavity sidewalls 72. Upon reaching the third position shown in FIG. 5, plunger head 29 will be about coextensive with lower handle open end 64. At this position, the tip member is dislodged and the button and plunger will have moved axially a second distance that is no greater than the axial extent of the handle connector walls 22.

Depending on the frictional engagement means being employed, it is likely the tip member will become loose and removable from the handle when the second distance has moved less than half the axial extent of connector walls 22.

FIGS. 1-5 illustrate cavity walls 72 that are flared outwardly for engagement with corresponding inwardly tapered connector walls 22. The wall surfaces are uniform and smooth whereby a forced friction engagement joint is created proximate the area adjacent flange 21. As such, only a short second distance movement will be required to disengage the tip member.

FIG. 9 illustrates an alternative tip member 30′ having a friction joint created by engagement of an annular rib 80 that extends inwardly from the inner surfaces of cavity walls 72′. A corresponding annular groove 82 is formed in the outer surfaces of connector walls 22′. In this embodiment, a snap fit is formed and released upon a predetermined axial movement of plunger 27. The location of the above joint structures along the axial extent of the connector wall and cavity wall, will dictate the aforementioned second distance.

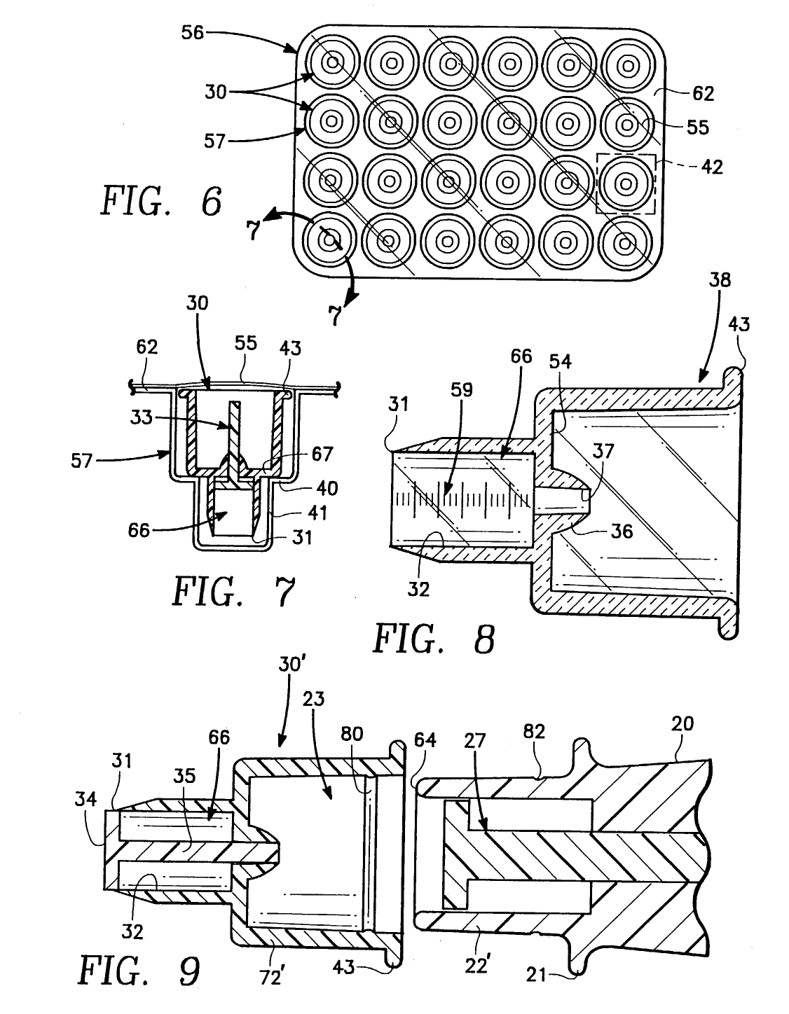

FIGS. 6 and 7 illustrate a package 56 of sterile disposable tip members 30. The package comprises a web 62 from which depend separate compartments 57 for each tip member. The tip members are oriented within each of their respective compartments 57 so that the open tip cavity 23 is uppermost.

It is also desirable to suspend cutting edge 31 above the bottom of the compartment. This is accomplished by providing an inwardly directed annular ledge 40 about the mid portion of compartment 57. The ledge functions to create a reduced diameter well 41 for constraining biopsy chamber 66. The well has a depth greater than the axial extent of the biopsy chamber, so that cutting edge 31 will be spaced above the compartment.

An impermeable cover 55 encloses each of the respective tip members within their respective compartments 57. The cover preferably comprises a heat sealable sheet of plastic, plastic coated foil, cellophane and the like which may be heat sealed or bonded to the web. Because an advantage of the present invention includes the elimination of laboratory sterilization costs by providing a sterile disposable tip to a user, the packaging cover and each tip member is sterilized and sealed to maintain a sealed sterile environment. Individual covers 42, depicted by phantom lines in FIG. 6, may also be used over each compartment. The covering is preferably clear.

According to one embodiment, prior to attaching a sterile tip member within a compartment to handle 10, the cover 55 is peeled back, exposing the sterile disposable tip within the compartment. The handle connector wall 22 is then pushed into tip cavity 23. This action will create the aforementioned friction joint and the tip member can then be lifted directly out of its compartment by movement of the handle without hand contact.

In an alternative embodiment, the handle connector wall may be positioned over cover 55, and forced into the tip cavity without removing the cover. As the handle is withdrawn, the cover will peel away from the package web 55 and become part of the friction joint.

There are multiple advantages to a process wherein the cover is not removed prior to attaching the handle. One is that the user need not waste time picking at a corner of a cover and trying to peel it back. Also, the presence of a thin sheet of cover material between the disposable tip member and the reusable plunger head 29, provides an additional barrier against fluid contamination from the tip member to the handle.

The package, covers and tip members can be sterilized by any known means. According to the preferred embodiment, they are sterilized through radiation sterilization procedures prior to shipping and distribution. The materials comprising the sterile tip and packaging are selected from materials that can withstand sterilization without experiencing undue degradation. Also, the materials used with reusable parts of the device can incorporate bactericide agents known in the art to further guard against contaminants.

Many variations of the foregoing description comprising equivalent structures and processes will be readily apparent to those skilled in the art as falling within the spirit and scope of the claimed invention. Accordingly, the appended claims are to be interpreted according to the widest scope of meaning.

What is claimed is:

1. A cutaneous biopsy device comprising a re-usable handle and a first quick-release disposable tip configured to removably couple to the handle, wherein the first quick-release disposable tip includes a cutting edge for cutting a cutaneous tissue sample of a first size.

2. The cutaneous biopsy device of claim 1 wherein the first quick-release disposable tip is configured to removably couple to the handle by a friction joint.

3. The cutaneous biopsy device of claim 2 wherein the quick-release disposable tip comprises a deformable member configured to deform when being coupled to the handle through the snap-fit.

4. The cutaneous biopsy device of claim 3 wherein the deformable member of the disposable tip is comprised of a first material, and the handle is comprised of a second material different than the first material.

5. The cutaneous biopsy device of claim 1 wherein the handle is further configured to couple to a second quick release disposable tip after the first quick release disposable tip has been disengaged from the handle, wherein the second quick-release disposable tip is configured to take a tissue sample of a different size than the first tissue sample.

6. The cutaneous biopsy device of claim 1 wherein the device is further configured to discharge the cutaneous tissue sample from the disposable tip by activation of a discharge mechanism integral to the cutaneous biopsy device.

7. The cutaneous biopsy device of claim 6 wherein the discharge mechanism for discharging the cutaneous tissue sample comprises a non-disposable plunger coupled to the handle and a disposable push rod coupled to the disposable tip, wherein the plunger is configured to engage the push rod when the non-disposable plunger is depressed, wherein the push rod is configured to engage the tissue sample and discharge the tissue sample from the removable tip.

8. The cutaneous biopsy device of claim 7 wherein the user has a thumb and fingers and the plunger includes a distal end that is ergonomically configured to be depressed by said thumb, wherein the handle further includes an ergonomic flange configured to engage at least one of said fingers to resist a force imparted by the user to the distal end of the plunger.

9. The cutaneous biopsy device of claim 8 wherein the ergonomic flange is an enlarged ring structure around the handle.

10. The cutaneous biopsy device of claim 8 wherein said distal end includes a button structure having an actuating head that is suitable for engagement with a user's hand.

11. The cutaneous biopsy device of claim 7 wherein the disposable tip further includes an abutment means configured to engage the plunger when the plunger is depressed a predetermined distance, and wherein a force imparted from the plunger onto the abutment means will separate the quick-release disposable tip from the handle.

12. The cutaneous biopsy device of claim 7 further comprising a resistive member configured to exert a resistive force against the plunger such that the plunger is returned to a non-depressed position when the plunger is not being depressed by a user.

13. The cutaneous biopsy device of claim 12 wherein the resistive member is a helical spring within the handle, wherein the helical spring is configured to exert a primary force against the plunger, and a secondary force opposing the primary force against the handle.

14. The cutaneous biopsy device of claim 1 wherein the disposable tip is configured to retain a cutaneous tissue sample within the disposable tip after the tissue sample has been cut.

15. The cutaneous biopsy device of claim 14 wherein the disposable tip is configured to retain the cutaneous tissue sample by cutting connective fatty tissue when the cutaneous biopsy device is rotated in a first position, swivelled to a second position, and rotated in the second position.

16. The cutaneous biopsy device of claim 1 wherein the first quick-release disposable tip includes a transparent portion.

17. The cutaneous biopsy device of claim 16 wherein the transparent portion of the disposable tip includes gradations that allow a user to determine a depth of tissue that has been cut by the cutaneous biopsy device.

18. The cutaneous biopsy device of claim 17 wherein the transparent portion includes the cutting edge, and wherein the transparent portion comprises a plastic material.

19. The cutaneous biopsy device of claim 18 wherein the cutting edge is formed in a mold without the use of machine tooling or abrasion to form the cutting edge.

20. A cutaneous biopsy apparatus for obtaining a cutaneous biopsy sample comprising:
a. disposable cutting means for severing a biopsy sample from cutaneous tissue and temporarily holding said sample;
b. reusable handle means for handling the cutting means;
c. coupling means for releasably coupling the disposable cutting means to the handle means;
d. discharge means for discharging the cutaneous tissue from the disposable cutting means, and
e. de-coupling means for dislodging the disposable cutting means from the reusable handle means.

21. The cutaneous biopsy apparatus of claim 20 wherein the de-coupling means comprises a manually activated plunger coupled to the re-usable handle means.

22. The cutaneous biopsy sample of claim 21 wherein the discharge means and the de-coupling means have a common component.

23. A method of configuring a cutaneous biopsy device comprising a handle having a discharge end and a first disposable quick release tip, wherein the tip is stored within a first compartment of a container covered by a sterile barrier, the method comprising the steps:
a. removing said sterile barrier configured to protect the first disposable quick-release tip enclosed in the first compartment;
b. positioning said discharge end of a handle against the first disposable quick-release tip;
c. inserting said discharge end into the first disposable quick-release tip; and
d. coupling the handle to the first disposable quick-release tip with a friction joint coupling, wherein the first disposable quick-release tip is configured to obtain a tissue sample of a first size.

24. The method according to claim 23 wherein the container comprises a plurality of compartments respectively storing a plurality of disposable quick-release tips individually protected by a respective plurality of sterile barriers.

25. The method according to claim 24 further comprising the step of withdrawing said first disposable quick-release tip from the container by moving said handle away from the container after step d.

26. The method according to claim 23 further comprising the steps:
e. activating a quick-release mechanism; and
f. releasing the first disposable quick-release tip from said handle.

27. The method according to claim 26 wherein the device includes a movable plunger within said handle, and the step of activating said quick-release mechanism comprises the steps:
g. depressing said plunger within the handle;
h. transmitting a force from said plunger to the first disposable quick-release tip.

28. The method according to claim 26 wherein said handle has a discharge end, further comprising the steps:
i. after step f., positioning said discharge end against a second disposable quick-release tip;
inserting said discharge end into the second disposable quick-release tip;
k. coupling the handle to the second disposable quick-release tip with a friction joint, wherein the second disposable quick-release tip is configured to obtain a tissue sample of a second size that may be similar to or distinct from said first size.

29. The method according to claim 23 further comprising the steps:
e. moving said first disposable quick-release tip into skin tissue until a selected tissue sample is lodged in said first disposable quick-release tip;
f. activating a tissue discharge assembly integral to the cutaneous biopsy device; and,
g. pushing the tissue sample out of the first disposable quick release tip.

30. The method according to claim 29 wherein said assembly comprises a plunger that is movable within said handle and a push rod that is movable within said first disposable quick release tip comprising the steps of:
o. moving said plunger against said push rod;
p. moving both the plunger and push rod in combination until the push rod engages said tissue sample; and,
q. moving the plunger, push rod and tissue sample in combination until said tissue sample is pushed out of said first disposable quick release tip.

31. A method of operating a cutaneous tissue-sampling device having a substantially round cutting edge formed on the edge of a substantially cylindrical member, the method comprising the steps:
a. pressing said cutting edge against a tissue, wherein the cutaneous tissue-sampling device is oriented on a conceptual plane at a reference angle of zero degrees;
b. rotating the cutaneous tissue-sampling device around an axis of the reference angle while maintaining pressure against the tissue;
c. moving the cutting edge into the tissue, thereby moving a tissue sample into the substantially cylindrical member;
d. angling the cutaneous tissue-sampling device about a positive angle of orientation on the conceptual plane;
e. rotating the cutaneous tissue-sampling device around an axis of the positive angle of orientation, thereby severing the tissue sample from underlying connective tissue; and
f. withdrawing the cutaneous tissue-sampling device, wherein the tissue sample remains lodged within the substantially cylindrical member of the tissue sampling device.

32. A sterile package of disposable tips for obtaining biopsy tissue samples comprising:
a plurality of compartments, each compartment containing a sterile disposable tip having a cutting edge and an engagement means for releasable attachment to a handle discharge end, said engagement means oriented in said compartment to receive said discharge end; and
a covering overlying each of said compartments.

33. The sterile package of claim 32 wherein a separate covering overlies each one of said compartments.

34. In a surgical cutting tool having a blade for cutting tissue wherein the improvement comprises constructing said blade of a transparent material.

35. The surgical cutting tool according to claim 34 wherein said blade includes gradations indicating a depth of tissue cut by the surgical cutting tool.

36. The surgical cutting tool according to claim 35 wherein said blade comprises a substantially cylindrical shape.

37. The surgical cutting tool according to claim 36 wherein said blade comprises a molded plastic material.

38. A skin biopsy device comprising:
a handle having a center passage and a discharge end;
a plunger positioned within said passage, said plunger having an actuating end and a contact end;
a tip member releasably attached to said discharge end, said tip member including a biopsy chamber having an outermost open end defined by a cutting edge and a push rod having a disc end in communication with said open end and a free end in communication with said plunger contact end.

39. In a skin biopsy device having a handle connected to a cutting edge, wherein the improvement comprises:
providing said cutting edge as part of a disposable tip member that is releasably connected to said handle.

40. The device of claim 39 wherein at least said cutting edge is constructed of plastic.

41. The device of claim 39 wherein said tip member is releasably connected to said handle by a friction joint.

42. The device of claim 41 wherein said handle has a movable plunger and said tip member has a push rod that is in communication with said plunger, said push rod being movable toward said cutting edge upon actuation from said plunger.

43. The device of claim 39 wherein at least a portion of said tip member proximate to said cutting edge is transparent.

44. The device of claim 42 wherein said handle has a central passage within which said plunger reciprocates, said tip member having an aperture within which said push rod moves, said plunger having a contact end that is engagable with said push rod to cause movement of said push rod within said aperture.

45. The device of claim 39 wherein said handle has a movable plunger for engaging said tip member and causing said tip member to disengage from said handle.

46. The device of claim 45 wherein said tip member has a tip cavity and said plunger has a contact end that extends into said cavity to disengage said tip member from said handle.

47. The device of claim 46 wherein said tip member includes a push rod that is movable toward said cutting edge upon actuation by said plunger prior to disengagement of said tip member from said handle.
